SEQUENCE LISTING (16 sequences)

SEQ ID NO: 1 - DNA, 20 bp, synthetic construct
  source 1..20: mol_type = other DNA; note = Primer ITS-5
agtcgtaacaaggtttccgt

SEQ ID NO: 2 - DNA, 19 bp, synthetic construct
  source 1..19: mol_type = other DNA; note = Primer ITS-3
tagtaacggcgagcgaacc

SEQ ID NO: 3 - DNA, 25 bp, synthetic construct
  source 1..25: mol_type = other DNA; note = Primer matK-5
cgtaccgtacttttatgtttacgag

SEQ ID NO: 4 - DNA, 27 bp, synthetic construct
  source 1..27: mol_type = other DNA; note = Primer matK-3
acccaatccatctggaaatcttgcttc

SEQ ID NO: 5 - DNA, 860 bp, Nymphaea sp.
  source 1..860: mol_type = genomic DNA; note = ITS28_Assembly
tagggagagcggccgccggatctcccggatggttcgagtttttcagcaagatgtcgtaacnaaggtttccgtaggtgaacctgcggaaggatcattgtcgtttccttttagatgacagacccgcgaacaagttatcattgctttcttcaagccgagcggagcatcgtttcccacgggaaagggtgatctgttctgcttcttggcatgtgcccgtcttttgccatcccctttgtggtcgattgcttgagcggtgtactgccaaaacaacaaaaacggcgcttttaagtgtcaaggatcatttattgaatgaaagggggacatcccgccacaaaatgagtggggggagaagtgcccctttgccttctaacaagaacgactctcggcaacggatatcttggctcctgtcacgatgaagaacgtagcgaaatgcgatagttggtgtgaattgcagaatcccgtgaatcatcgagtttttgaacgcaagttgcgcccgaggccattcggccaagggcacgtctgcctgggcgtcacgcttagcgtcgccctccccaggtcctcgtgttttcgaaccgaggccgagggagagcggaggactggccttcggtgtcgctttcatcggcgtcgtcggctgaaactttcggctcacgatctgttgtgcagcacaacaagcggtggatttccagtgagttgttgtgtttcacgtggtcgaagggccatgggactcgaggcaaggttctcattttcttgccttagctttgcgaccccaggtcaggcgagactacccgctgagtttaagcatatcaataagcggatctctctagcaggtctcctacaatattctcagctgccatggaaaatcg

SEQ ID NO: 6 - DNA, 857 bp, Nymphaea sp.
  source 1..857: mol_type = genomic DNA; note = ITS40_Assembly
ttcgattttccatggcagctgagaatattgtaggagactgctagagagatagtcgtaacaatttccgtaggtgaacctgcggaaggatcattgtcgtttccttagatgacagacccgcgaacaagttatcattgctttcttcaagccgagcggagcatcgtttcccacgggaaagggtgatctgttctgcttcttggcatgtgcccgtcttttgccatcccctttgtggtcgattgcttgagcggtgtactgccaaaacaacaaaaacggcgcttttaagtgtcaaggatcatttattgaatgaaagggggacatcccgccacaaaatgagtggggggagaagtgcccctttgccttctaacaagaacgactctcggcaacggatatcttggctcctgtcacgatgaagaacgtagcgaaatgcgatagttggtgtgaattgcagaatcccgtgaatcatcgagtttttgaacgcaagttgcgcccgaggccattcggccaagggcacgtctgcctgggcgtcacgcttagcgtcgccctccccaggtcctcgtgttttcgaaccgaggccgagggagagcggaggactggccttcggtgtcgctttcatcggcgtcgtcggctgaaactttcggctcacgatctgttgtgcagcacaacaagcggtggatttccagtgagttgttgtgtttcacgtggtcaaagggccatgggactcgaggcaaggttctcattttcttgccttagctttgcgaccccaggtcaggcgagactacccgctgagtttaagcatatctngctgaaaaactcgaaccatcccgggagatcccggcggccgctctccctataggtgagtc

SEQ ID NO: 7 - DNA, 860 bp, Nymphaea sp.
  source 1..860: mol_type = genomic DNA; note = ITS88_Assembly
ttttccatggcagctgagaatatgtaggagacctgctagagagatagtcgtaacaaggtttccgtaggtgaacctgcggaaggatcattgtcgtttccttttagatgacagacccgcgaacaagttatcattgctttcttcaagccgagcggagcatcgtttcccacgggaaagggtgatctgttctgcttcttggcatgtgcccgtcttttgccatcccctttgtggtcgattgcttgagcggtgtactgccaaaacaacaaaaacggcgcttttaagtgtcaaggatcatttattgaatgaaagggggacatcccgccacaaaatgagtggggggagaagtgcccctttgccttctaacaagaacgactctcggcaacggatatcttggctcctgtcacgatgaagaacgtagcgaaatgcgatagttggtgtgaattgcagaatcccgtgaatcatcgagtttttgaacgcaagttgcgcccgaggccattcggccaagggcacgtctgcctgggcgtcacgcttagcgtcgccctccccaggtcctcgtgttttcgaaccgaggccgagggagagcggaggactggccttcggtgtcgctttcatcggcgtcgtcggctgaaactttcggctcacgatctgttgtgcagcacaacaagcggtggatttccagtgagttgttgtgcttcgcgtgatcgaagggccacgggactcgaggcaaggagctcagtttcttgcccaagctttgcgaccccaggtcaggcgagactacccgctgagtttaagcatatcaataagcggaggaatcttgctgaaaaactcgaaccatccgggagatccggcggccgctctcccta

SEQ ID NO: 8 - DNA, 855 bp, Nymphaea sp.
  source 1..855: mol_type = genomic DNA; note = ITS115_Assembly
ttccatggcagctgagaatattgtaggagactgctagagagatagtcgtaacaatttccgtaggtgaacctgcggaaggatcattgtcgtttccttagaggacagacccgcgaacatgttatcattgctttcttcaagccgggcggtgcatcgtgccccacggggcaggttgatccgttctgcttcttggcatgtgcccgtcttttgccatcccctttactgtggtcgatggtttgagcggcgtattgccaaaacaataaaaacggcgcttttaagtgtcaaggatcatttattgaatgaaagggggacatcccgccacaaaatgagtggggggagaagtgcccctttgccttctaacaagaacgactctcggcaacggatatcttggctcctgtcacgatgaagaacgtagcgaaatgcgatagttggtgtgaattgcagaatcccgtgaatcatcgagtttttgaacgcaagttgcgcccgaggccattcggccaagggcacgtctgcctgggcgtcacgcttagcgtcgccctccccaggtcctcgtgttttcgaaccgaggccgagggagagcggaggactggccttcggtgtcgctttcatcggcgtcgtcggctgaaactttcggctcacgatctgttgtgcagcacaacaagcggtggatttccagtgagttgttgtgtttcacgtggtcaaagggccatgggactcgaggcaaggttctcattttcttgccttagctttgcgaccccaggtcaggcgagactacccgctgagtttaagcatatcaataagcggaggatcttgctgaaaaactcgaaccatccgggagatccggcggccgctctccta

SEQ ID NO: 9 - DNA, 856 bp, Nymphaea sp.
  source 1..856: mol_type = genomic DNA; note = ITS142_Assembly
tagggagagcggccgccggatctcccggatggttcgagtttttcagcaagatagtcgtaacaatttccgtaggtgaacctgcggaaggatcattgtcgtttccttagatgacagacccgcgaacaagttatcattgctttcttcaagccgagcggagcatcgtttcccacgggaaagggtgatctgttctgcttcttggcatgtgcccgtcttttgccatcccctttgtggtcgattgcttgagcggtgtactgccaaaacaacaaaaacggcgcttttaagtgtcaaggatcatttattgaatgaaagggggacatcccgccacaaaatgagtggggggagaagtgcccctttgccttctaacaagaacgactctcggcaacggatatcttggctcctgtcacgatgaagaacgtagcgaaatgcgatagttggtgtgaattgcagaatcccgtgaatcatcgagtttttgaacgcaagttgcgcccgaggccattcggccaagggcacgtctgcctgggcgtcacgcttagcgtcgccatccccaggtcctcgtgtttccgaactgaggccgagggagagcggaggactggccttcggtgtcgtcggcgtcgtcggctgaaacttttggctcgcgatctgttgtgcggcacaacaagcggtggatttccagtgagttgttgtgcttcgcgtgatcgaagggccacgggactcgaggcaaggagctcagtttcttgcccaagctttgcgaccccaggtcaggcgagactacccgctgagtttaagcatatcaataagcggaggaatctctctagcaggtctcctacaatattctcagctgccatggaaaaatcga

SEQ ID NO: 10 - DNA, 886 bp, Nymphaea sp.
  source 1..886: mol_type = genomic DNA; mol_type = genomic DNA; note = ITS180_Assembly
tcaaaaaacatcgattttccatggcagctgagaatattgtaggagacctgctagagagatagtcgtaacaatttccgtaggtgaacctgcggaaggatcattgtcgtttccttagaggacagacccgcgaacatgttatcattgctttcttcaagccgggcggtgcatcgtgccccacggggcaggttgatccgttctgcttcttggcatgtgcccgtcttttgccatcccctttactgtggtcgatggtttgagcggcgtattgccaaaacaataaaaccggcgcttttaagcgtcaaggatcattgattgaatgaaagggggacatcctgccacaaatgagtggggggagaagtgcccctttgccttctaataagaacgactctcggcaacggatatcttggctcccgtcacgatgaagaacgtagcgaaatgcgatagttggtgtgaattgcagaatcccgtgaatcatcgagtttttgaacgcaagttgcgcccgaggccattcggccaagggcacgtctgcctgggcgtcacgcttagcgtcgccatccccaggtcctcgtgtttccgaactgaggccgagggagagcggaggactggccttcggtgtcgctttcatcggcgtcgtcggctgaaactttcggctcacgatctgttgtgcagcacaacaagcggtggatttccagtgagttgttgtgtttcacgtggtcaaagggccatgggactcgaggcaaggttctcattttcttgccttagctttgcgaccccaggtcaggcgagactacccgctgagtttaagcatatcaataagcggagatcttgctgaaaaactcgaaccatccmgkagatcccggcggccgctctccctataggtggagtcaga

SEQ ID NO: 11 - DNA, 866 bp, Nymphaea sp.
  source 1..866: mol_type = genomic DNA; note = ITS198_Assembly
aaaggaacacactgcgtttccatggcagctgagaatattgtaggagactgctagagagatagtcgtaacaatttccgtaggtgaacctgcggaaggatcattgtcgtttccttagaggacagacccgcgaacatgttatcattgctttcttcaagccgggcggtgcatcgtgccccacggggcaggttgatccgttctgcttcttggcatgtgcccgtcttttgccatcccctttactgtggtcgatggtttgagcggcgtattgccaaaacaataaaaccggcgcttttaagcgtcaaggatcattgattgaatgaaagggggacatcctgccacaaatgagtggggggagaagtgcccctttgccttctaataagaacgactctcggcaacggatatcttggctcccgtcacgatgaagaacgtagcgaaatgcgatagttggtgtgaattgcagaatcccgtgaatcatcgagtttttgaacgcaagttgcgcccgaggccattcggccaagggcacgtctgcctgggcgtcacgcttagcgtcgccatccccaggtcctcgtgtttccgaattgaggccgagggagagcggaggactggccttcggtgtcgtcggcgtcgtcggctgaaacttttggctcgcgatctgttgtgcggcacaacaagcggtggatttccagtgagttgttgtgcttcgcgtgatcgaagggccacgggactcgaggcaaggagctcagtttcttgcccaagctttgcgaccccaggtcaggcgagactacccgctgagtttaagcatatcaataagcggaggatcttgctgaaaaactcgaaccatccgggagatccggcggccgctctccttt

SEQ ID NO: 12 - DNA, 881 bp, Nymphaea sp.
  source 1..881: mol_type = genomic DNA; note = ITS212_Assembly
tcgactcacctatagggagagcggccgccggatctcccggatggttcgagtttttcagcaagatagtcgtaacaatttccgtaggtgaacctgcggaaggatcattgtcgtttccttagatgacagacccgcgaacaagttatcattgctttcttcaagccgagcggagcatcgtttcccacgggaaagggtgatctgttctgcttcttggcatgtgcccgtcttttgccatcccctttgtggtcgattgcttgagcggtgtactgccaaaacaacaaaaacggcgcttttaagtgtcaaggatcatttattgaatgaaagggggacatcccgccacaaaatgagtggggggagaagtgcccctttgccttctaacaagaacgactctcggcaacggatatcttggctcctgtcacgatgaagaacgtagcgaaatgcgatagttggtgtgaattgcagaatcccgtgaatcatcgagtttttgaacgcaagttgcgcccgaggccattcggccaagggcacgtctgcctgggcgtcacgcttagcgtcgccctccccaggtcctcgtgttttcgaaccgaggccgagggagagcggaggactggccttcggtgtcgtcggcgtcgtcggctgaaacttttggctcgcgatctgttgtgcggcacaacaagcggtggatttccagtgagttgttgtgcttcgcgtgatcgaagggccacgggactcgaggcaaggagctcagtttcttgcccaagctttgcgaccccaggtcaggcgagactacccgctgagtttaagcatatcaataagcggaggaatctctctagcagtctcctacaatattctcagctgccatggaaatcgaaatggtttctttataa

SEQ ID NO: 13 - DNA, 936 bp, Nymphaea sp.
  source 1..936: mol_type = genomic DNA; note = ITS213_Assembly
ctaaagagaacactattgcgatttccatggcagctgagaatatgtaggagactgctagagagatagtcgtaacaaggtttccgtaggtgaacctgcggaaggatcattgtcgtttccttttagatgacagacccgcgaacaagttatcattgctttcttcaagccgagcggagcatcgtttcccacgggaaagggtgatctgttctgcttcttggcatgtgcccgtcttttgccatcccctttgtggtcgattgcttgagcggtgtactgccaaaacaacaaaaacggcgcttttaagtgtcaaggatcatttattgaatgaaagggggacatcccgccacaaaatgagtggggggagaagtgcccctttgccttctaacaagaacgactctcggcaacggatatcttggctcctgtcacgatgaagaacgtagcgaaatgcgatagttggtgtgaattgcagaatcccgtgaatcatcgagtttttgaacgcaagttgcgcccgaggccattcggccaagggcacgtctgcctgggcgtcacgcttagcgtcgccctccccaggtcctcgtgttttcgaaccgaggccgagggagagcggaggactggccttcggtgtcgctttcatcggcgtcgtcggctgaaactttcggctcacgatctgttgtgcagcacaacaagcggtggatttccagtgagttgttgtgtttcacgtggtcaaagggccatgggactcgaggcaaggttctcattttcttgccttagctttgcgaccccaggtcaggcgagactacccgctgagtttaagcatatcaataagcggaggaaaagaaacttacaaggattcccctagtaacggcgagcgaaccatcttgctgaaaaactcgaaccatcccgggagatcccggcggccgctctccctataggtgagtcgaa

SEQ ID NO: 14 - DNA, 889 bp, Nymphaea sp.
  source 1..889: mol_type = genomic DNA; note = ITS258_Assembly
tcgaaagaaccattcgattttcccatggcagctgagaatattngtaggagacctgctagagagatagtcgtaacaatttccgtaggtgaacctgcggaaggatcattgtcgtttccttagaggacagacccgcgaacatgttatcattgctttcttcaagccgggcggtgcatcgtgccccacggggcaggttgatctgttctgcttcttggcatgtgcccgtcttttgccatcccctttgtggtcgattgcttgagcggtgtactgccaaaacaacaaaaacggcgcttttaagtgtcaaggatcatttattgaatgaaagggggacatcccgccacaaaatgagtggggggagaagtgcccctttgccttctaacaagaacgactctcggcaacggatatcttggctcctgtcacgatgaagaacgtagcgaaatgcgatagttggtgtgaattgcagaatcccgtgaatcatcgagtttttgaacgcaagttgcgcccgaggccattcggccaagggcacgtctgcctgggcgtcacgcttagcgtcgccctccccaggtcctcgtgttttcgaaccgaggccgagggagagcggaggactggccttcggtgtcgctttcatcggcgtcgtcggctgaaactttcggctcacgatctgttgtgcagcacaacaagcggtggatttccagtgagttgttgtgtttcacgtggtcaaagggccatgggactcgaggcaaggttctcattttcttgccttagctttgcgaccccaggtcaggcgagactacccgctgagtttaagcatatcaataagcggaggatcttgctgaaaaactcgaaccnatccsggagatccggcggccgctctccctatagggtgagtcga

SEQ ID NO: 15 - DNA, 1008 bp, Nymphaea sp.
  source 1..1008: mol_type = genomic DNA; note = matK8_Assembly
atttccctttttagaggacaaattatcacatttatattatgtttcagatatactaataccctacccaatccatctggaaatcttgcttcaaactcttcgcactcggatacgagatgctccttctttgcatttattgagatgttttctacatgagcatcataattggaatagccttattacttctacttcaaataaatccatttccattttttcaaaggaaaatcaaagattattcttgttcttgtataattctcatgtatatgaatgcgaatccgtattagttttccttcgtaaacaatcctctcatttacggtcaatatcttctctagcctttcttgagagaacacatttttatggaaaaataaaacatcttgtagtgacgcctcgtaatgattctcaaaggaccctgcccctctggttcttcaaagaacctttgatgcattatgttaggtatcaaggaaaatcaattatggcttcaaggtgtactaatttactgatgaagaaatggaaatattaccttgtcaatttctggcaatgtcattttcacttatggtctcaaccgggtaggatccatataaatgaattatccaatcattctttctattttctgggctatcttttaggtgtacgactaacgccttgggtgataaggagtcaaatgctagagaattcatttatgatcgatactgctattaagagattcgatacaatagtcccaatttttcctctgattggatcgttggttaaagctaaattctgtaacgtatcagggtatcctattagtaagtcagtctgggccgattcgtcggattctgatattattgctcgattcgggtggatatgcagaaatctctctcattatcacagcggatcctcaaaaaaacacagtttgtgtcgaataaagtatatacttcgactttcgtgtgctagaactctagctcgtaaacataaaagtacggtacgcgcaatctgtaagagattaggttcaaaactattggaagagttcct

SEQ ID NO: 16 - DNA, 977 bp, Nymphaea sp.
  source 1..977: mol_type = genomic DNA; note = matK11_Assembly
atactaataccctacccaatccatctggaaatcttgcttcaaactcttcgcactcggatacgagatgctccttctttgcatttattgagatgttttctacatgagcatcataattggaatagccttattacttctacttcaaataaatccatttccattttttcaaaggaaaatcaaagattattcttgttcttgtataattctcatgtatatgaatgcgaatccgtattagttttccttcgtaaacaatcctctcatttacggtcaatatcttctctagcctttcttgagagaacacatttttatggaaaaataaaacatcttgtagtgacgcctcgtaatgattctcaaaggaccctgcccctctggttcttcaaagaacctttgatgcattatgttaggtatcaaggaaaatcaattatggcttcaaggtgtactaatttactgatgaagaaatggaaatattaccttgtcaatttctggcaatgtcattttcacttatggtctcaaccgggtaggatccatataaatgaattatccaatcattctttctattttctgggctatcttttaggtgtacgactaacgccttgggtgataaggagtcaaatgctagagaattcatttatgatcgatactgctattaagagattcgatacaatagtcccaatttttcctctgattggatcgttggttaaagctaaattctgtaacgtatcagggtatcctattagtaagtcagtctgggccgattcgtcggattctgatattattgctcgattcgggtggatatgcagaaatctctctcattatcacagcggatcctcaaaaaaacacagtttgtgtcgaataaagtatatacttcgactttcgtgtgctagaactctagctcgtaaacataaaagtacggtacgcgcaatctgtaagagattaggttcaaaactattggaagagttccttacagaggaacaagaaat

(19) United States
(12) Patent Application Publication
(10) Pub. No.: US 2023/0407416 A1
(43) Pub. Date: Dec. 21, 2023

(54) IDENTIFICATION METHOD OF TWO PARENTS OF NYMPHAEA HYBRID BASED ON SEQUENCES OF INTERNAL TRANSCRIBED SPACER (ITS) AND matK

(71) Applicant: Kunming Institute of Botany, CAS, Kunming City (CN)

(72) Inventors: Zhengshan He, Kunming City (CN); Junbo Yang, Kunming City (CN); Tingshuang Yi, Kunming City (CN); Jie Cai, Kunming City (CN); Zhenyan Yang, Kunming City (CN); Chunxia Zeng, Kunming City (CN); Jixiong Yang, Kunming City (CN); Jing Yang, Kunming City (CN); Zhirong Zhang, Kunming City (CN); Dezhu Li, Kunming City (CN)

(73) Assignee: Kunming Institute of Botany, CAS, Kunming City (CN)

(21) Appl. No.: 18/318,565

(22) Filed: May 16, 2023

(30) Foreign Application Priority Data
May 17, 2022 (CN) 202210533264.8

(51) Int. Cl. C12Q 1/6895; G16B 50/00; G16B 30/10

(52) U.S. Cl. CPC C12Q 1/6895; G16B 30/10; G16B 50/00

(57) ABSTRACT

The present disclosure provides an identification method of two parents of a Nymphaea hybrid based on sequences of internal transcribed spacer (ITS) and matK, and belongs to the technical field of plant molecular identification. In the present disclosure, the identification method includes: based on Sanger sequencing, obtaining an ITS sequence of a nuclear genome fragment inherited from the two parents and a matK sequence of a chloroplast genome fragment inherited from a maternal line in the Nymphaea hybrid; subjecting ITS and matK sequences downloaded from a GenBank to strict screening, and establishing a database of the ITS and matK sequence of the Nymphaea; aligning ITS and matK sequences of the Nymphaea of the parents to be determined with respective database, and constructing a neighbor-joining tree based on a genetic distance; and checking specific loci to obtain information on male and female parents of the Nymphaea to be determined.

### CROSS REFERENCE TO RELATED APPLICATION

This application claims the benefit and priority to Chinese Patent Application No. 202210533264.8, filed May 17, 2022, the content of which is incorporated herein by reference in its entirety.

### REFERENCE TO SEQUENCE LISTING

A computer readable txt. file entitled “GWP20230402643”, that was created on May 16, 2023, with a file size of about 28,615 bytes, contains the sequence listing for this application, has been filed with this application, and is hereby incorporated by reference in its entirety.

### TECHNICAL FIELD

The present disclosure belongs to the technical field of plant molecular identification, and specifically relates to an identification method of two parents of a Nymphaea hybrid based on sequences of internal transcribed spacer (ITS) and matK. The identification method combines an ITS nucleotide sequence of a nuclear DNA fragment and a matK nucleotide sequence of a chloroplast DNA fragment to conduct molecular identification on the Nymphaea hybrid (by natural or artificial hybridization, referring to Chapter I Article 1 of the Ninth Edition of the International Code of Botanical Nomenclature in 2016).

### BACKGROUND

Water lily, as a general term for plants of the genus Nymphaea of the family Nymphaeaceae, is widely distributed all over the world. There are about 50 species of Nymphaea, in which 5 species are produced in China. Nymphaea can be divided into 5 subgenuses, and can also be divided into cold-resistant types and tropical types according to ecological adaptability. Nymphaea has a wide variety and rich colors. The Nymphaea is thus known as a “Pond Palette” and has been cultivated for as long as 4,000 years. The root Nymph of a Latin genus name of Nymphaea is the elf fairy in mountains, forests, and waters in Greek mythology. There are about 2,000 species in the Nymphaea worldwide (http ://www.victoria-adventure.org/waterlilies/names/names_a_z.htm). Nymphaea has rich flower colors, long flowering period, and strong adaptability and stress resistance, and is easy to cultivate. Moreover, the Nymphaea has flower-viewing varieties of various flower colors, colorful foliage-viewing varieties, and fragrant and pleasant perfume-enjoying varieties. Nymphaea is the national flower of countries such as Egypt, India, Bangladesh, and Sri Lanka, and has a long history of utilization. In addition to important ornamental values, Nymphaea also has important cultural and economic values. In the Nymphaea, leaves, petioles, pedicels, and flowers are tropical regular vegetables, and tubers can be made into edible starch. Meanwhile, the Nymphaea is rich in various polyphenols and flavones, thus showing a certain medicinal value (SELVAKUMARI et al., 2016). Ecologically, Nymphaea can also be used to purify water bodies (ZIARATI et al., 2015). The related industries such as new variety breeding, food, and health care of Nymphaea have begun to take shape at home and abroad (Li Shujuan et al., 2019). The Nymphaea is widely cultivated. However, many hybrid varieties lack complete recorded information of hybridization, with their parents unidentified. This poses challenges for variety identification and further cross breeding.

Different from morphological identification, molecular identification can obtain specific characteristics that are difficult to obtain in morphology from the DNA sequence information of a species, thereby identifying species and varieties that are difficult to distinguish by the morphological identification. In angiosperms, the vast majority of chloroplast genomes are uniparentally and maternally inherited, while nuclear genes are biparentally inherited (O'KANE et al., 1996). The internal transcribed spacer (ITS) sequence in a nuclear genome is the ITS fragment in a ribosomal DNA (rDNA). Eukaryotic ribosomes are composed of ribosomal RNA (rRNA) and ribosomal protein, and the rRNA is encoded by the rDNA. The rRNA of 18S, 5.8S, and 25S (S represents a sedimentation coefficient) in plants is transcribed by an rDNA transcription unit of 45S with the assistance of polymerase I. Plants have two ITS sequences, ITS1 and ITS2 (both about 240 bp in length), where the ITS1 is located between 18S rRNA and 5.8S rRNA, and the ITS2 is located between 5.8S rRNA and 25S rRNA. Many species of hybrid origin retain variation sites of the ITS sequences of their parents. A matK gene in the chloroplast genome shows a high evolution rate and easy amplification, and has also been widely used in plant identification of barcode technology.

At present, there is no report on an identification method of two parents of a Nymphaea hybrid based on sequences of ITS and matK in the prior art.

### SUMMARY

An objective of the present disclosure is to provide an identification method of two parents of a Nymphaea hybrid based on sequences of ITS and matK. A problem to be solved by the present disclosure is that there is a lack of an effective identification method of two parents of a Nymphaea hybrid in the prior art.

To achieve the above objective, the present disclosure provides the following technical solutions:

The present disclosure provides an identification method of two parents of a Nymphaea hybrid based on sequences of ITS and matK, including the following steps: constructing a database for each of nucleotide sequences of ITS and matK of Nymphaea, and designing an applicable primer of the Nymphaea to conduct PCR amplification; aligning sequences of ITS and matK obtained by monoclonal sequencing with the respective database; constructing a neighbor-joining tree based on a genetic distance, and checking specific loci to obtain species information of male and female parents of the Nymphaea hybrid.

The present disclosure provides an identification method of two parents of a Nymphaea hybrid based on sequences of ITS and matK, including the following steps:

construction of a database for each of nucleotide sequences ITS and matK of Nymphaea: download existing nucleotide sequences of ITS and matK of Nymphaea; conducting stringent filtration to retain one sequence for each “species unit”, namely species/subspecies/variety/form/hybrid of the sequences of ITS and matK; conducting alignment, adjusting sequences with inconsistent sequence directions, and then conducting re-alignment to obtain the database for each of nucleotide sequences ITS and matK of Nymphaea; and arranging a sequence name for each of finally obtained sequences in a format of “species name/ACCESSION of sequence”; and

construction of an ITS monoclonal sequence and an matK nucleotide sequence of a species for parents to be determined of the Nymphaea: extracting a genomic DNA of the Nymphaea of the parents to be determined; designing primers; conducting PCR sequence amplification; conducting Sanger sequencing; conducting sequence alignment and construction of a neighbor-joining tree, and then aligning sequences of ITS and matK obtained by monoclonal sequencing with the respective database; constructing a neighbor-joining tree based on a genetic distance, and checking specific loci to obtain species information of male and female parents of the Nymphaea hybrid.

In the present disclosure, a process of construction of the database for each of the nucleotide sequences ITS and matK of the Nymphaea specifically includes:
a, searching a nucleotide database of the National Center of Biotechnology Information (NCBI, www.ncbi.nlm.nih.gov/nucleotide) with syntaxes “((Nymphaea[Organism]) AND 5.8S[Title]) NOT PREDICTED[Title]” and “((Nymphaea[Organism]) AND matK[Title]) NOT PREDICTED[Title]”, to obtain all data of the sequences of ITS and matK of the Nymphaea; acquiring 59 published chloroplast genomes of the Nymphaea with a syntax “chloroplast, complete genome[Title] OR plastid, complete genome[Title]) AND Nymphaea[Organism]”, and extracting a matK sequence in the chloroplast genomes;
b, conducting stringent filtration to retain one sequence for each “species unit”, namely species/subspecies/variety/form/hybrid of the sequences of ITS and matK; and
c, conducting alignment, adjusting the sequences with inconsistent sequence directions, and then conducting re-alignment to obtain the database of the nucleotide sequences ITS and matK of the Nymphaea.

In the present disclosure, the stringent filtration specifically includes:
a, removing a species whose species name includes unverified, sp., and cf , retaining a subspecies, a variety, a form, and a clear hybrid that are included in the species, which are referred to as “species units”, and regarding each of an isolate, a voucher, a genotype, and a strain as a cultivar and classifying the cultivar into each “species unit”;
b, if the “species unit” has only one sequence, omitting the filtration; after the filtration is conducted, retaining only one sequence for each “species unit”;
c, if a sequence of the cultivar in step a is quite different from that of the “species unit”, removing the sequence of the cultivar preferentially;
d, removing a sequence with a significantly high difference, especially in a coding region such as 5.8S rRNA preferentially; removing a sequence having an unknown base and a degenerate base preferentially; and retaining a longer sequence preferentially;
e, using the matK sequence extracted from the chloroplast genomes only as a supplement; when the matK sequence obtained by sequencing already has the “species unit”, discarding the matK sequence extracted from the chloroplast genomes preferentially; and
f, retaining a sequence as a sequence that best represents the “species unit”, that is, a sequence being closest to a consensus sequence obtained from multiple sequences.

Further, a process of the construction of an ITS monoclonal sequence and an matK nucleotide sequence of a species for parents to be determined of the Nymphaea specifically includes:
a, designing an ITS primer suitable for the Nymphaea using conserved nucleotide sequences of 18S, 5.8S, and 25S rRNA of the Nymphaea and referring to a general plant barcode ITS primer; and designing a matK primer suitable for the Nymphaea using a conserved nucleotide sequence of matK of the Nymphaea and referring to a general plant barcode primer;
b, extracting the genomic DNA of the Nymphaea of the parents to be determined;
c, conducting PCR amplification using the genomic DNA of the Nymphaea of the parents to be determined obtained in step b as a template and using the primers obtained in step a, to obtain amplification products of the sequences of ITS and matK, respectively;
d, ligating the amplification product of the ITS sequence obtained in step c using a pLB zero-background rapid cloning kit, and transforming into DH5α competent cells; conducting PCR amplification on an obtained selected bacterial plaque through a carrier primer, to obtain a PCR amplification product of the ITS sequence by cloning amplification; and
e, subjecting the amplification product of the matK sequence obtained in step c and the PCR amplification product of the ITS sequence by cloning amplification obtained in step d to sequencing with a Sanger sequencer, and conducting sequence assembly on obtained sequencing results to obtain the matK sequence and the monoclonal ITS sequence for the parents to be determined of the Nymphaea.

Further, the primers include:

ITS-5, with a nucleotide sequence of
(SEQ ID NO: 1)
AGTCGTAACAAGGTTTCCGT;
ITS-3, with a nucleotide sequence of
(SEQ ID NO: 2)
TAGTAACGGCGAGCGAACC;
matK-5, with a nucleotide sequence of
(SEQ ID NO: 3)
CGTACCGTACTTTTATGTTTACGAG;
and
matK-3, with a nucleotide sequence of
(SEQ ID NO: 4)
ACCCAATCCATCTGGAAATCTTGCTTC.

Further, a reaction system of the PCR amplification includes:

a PCR amplification system of the ITS sequence is 30 μL, including 15 μL of PCR 2× Mix, 13 μL of ddH₂O, 0.5 μL for each of the primers, and 1.0 μL of a total DNA; the PCR amplification of the ITS sequence includes the following procedures: initial denaturation at 95° C. for 3 min; denaturation at 94° C. for 55 s, annealing at 55° C. for 55 s, and extension at 72° C. for 70 s, conducting 35 cyles; and extension at 72° C. for 7 min; a PCR amplification system of the matK sequence is 20 μL, including 10 μL of the PCR 2× Mix, 8 μL of the ddH₂O, 0.5 μL for each of the primers, and 1.0 μL of the total DNA; and the PCR amplification of the matK sequence includes the following procedures: initial denaturation at 95° C. for 3 min; denaturation at 94° C. for 30 s, annealing at 52° C. for 30 s, and extension at 72° C. for 1 min, conducting 35 cyles; and extension at 72° C. for 5 min.

Further, a reaction system of the PCR amplification for a pLB-ligated ITS clone product includes: an amplification system is 20 μL, including 10 μL of PCR 2× Mix, 10 μL of ddH₂O, 0.3 μL for each of the primers, and 1 cluster of a pLB-ligated bacterial plaque; the PCR amplification includes the following procedures: initial denaturation at 95° C. for 3 min; denaturation at 94° C. for 55 s, annealing at 55° C. for 55 s, and extension at 72° C. for 80 s, conducting 35 cycles; and extension at 72° C. for 7 min.

Further, a reaction system of the sequencing includes: a sequencing reaction of the ITS and the matK have same reaction system and reaction conditions; the reaction system is 6 μL, including 0.3 μL of Bigdye, 1.0 μL of SeqBuffer, 3.70 μL of ddH₂O, 0.5 μL of a single-end primer, and 0.5 μL of a PCR purified product; and the reaction conditions include: initial denaturation at 94° C. for 30 s; denaturation at 96° C. for 10 s, annealing at 50° C. for 5 s, and extension at 60° C. for 3 min, conducting 32 cycles.

In the present disclosure, the identification method can be summarized as follows:

In the present disclosure, a process of construction of the database for each of the nucleotide sequences ITS and matK of the Nymphaea includes: a, searching a nucleotide database of the National Center of Biotechnology Information (NCBI, www.ncbi.nlm.nih.gov/nucleotide) with syntaxes “((Nymphaea[Organism]) AND 5.8S [Title]) NOT PREDICTED[Title]” and “((Nymphaea[Organism]) AND matK[Title]) NOT PREDICTED[Title]”, to obtain all data of the sequences of ITS and matK of the Nymphaea; acquiring 59 published chloroplast genomes of the Nymphaea with a syntax “chloroplast, complete genome[Title] OR plastid, complete genome[Title]) AND Nymphaea[Organism]” , and extracting a matK sequence in the chloroplast genomes; b, conducting stringent filtration to retain one sequence for each “species unit”, namely species/subspecies/variety/form/hybrid of the sequences of ITS and matK; and c, conducting alignment, adjusting the sequences with inconsistent sequence directions, and then conducting re-alignment to obtain the database of the nucleotide sequences ITS and matK of the Nymphaea.

To obtain the ITS and matK sequences of the Nymphaea to be determined, the genomic DNA of the Nymphaea of the parents to be determined is extracted, and then the conserved nucleotide sequences of 18S, 5.8S, and 25S rRNA of the Nymphaea are used to design ITS primers applicable to Nymphaea referring to the ITS universal primers of plant barcodes. That is, the primer pair includes: a forward primer ITS-5 (AGTCGTAACAAGGTTTCCGT) (SEQ ID NO:1) and a reverse primer ITS-3 (TAGTAACGGCGAGCGAACC) (SEQ ID NO:2). The conserved nucleotide sequences of matK of the Nymphaea are used to design matK primers applicable to Nymphaea referring to the universal primers of plant barcodes. That is, the primer pair includes: a forward primer matK-5 (CGTACCGTACTTTTATGTTTACGAG) (SEQ ID NO:3) and a reverse primer matK-3 (ACCCAATCCATCTGGAAATCTTGCTTC) (SEQ ID NO:4). A PCR amplification product of the ITS is obtained by the primer pair ITS-5/ITS-3, and a PCR amplification product of the matK is obtained by the primer pair matK-5/matK-3; a PCR amplification system of the ITS sequence is 30 μL, including 15 μL of PCR 2× Mix, 13 μL of ddH₂O, 0.5 μL for each of the primers, and 1.0 μL of a total DNA; the PCR amplification of the ITS sequence includes the following procedures: initial denaturation at 95° C. for 3 min; denaturation at 94° C. for 55 s, annealing at 55° C. for 55 s, and extension at 72° C. for 70 s, conducting cyles; and extension at 72° C. for 7 min; a PCR amplification system of the matK sequence is 20 μL, including 10 μL of the PCR 2× Mix, 8 μL of the ddH₂O, 0.5 μL for each of the primers, and 1.0 μL of the total DNA; and the PCR amplification of the matK sequence includes the following procedures: initial denaturation at 95° C. for 3 min; denaturation at 94° C. for 30 s, annealing at 52° C. for 30 s, and extension at 72° C. for 1 min, conducting 35 cyles; and extension at 72° C. for 5 min.

d, after the PCR amplification product of the ITS is obtained with the primer pair ITS-5/ITS-3, ligating the amplification product of the ITS sequence obtained in step c then using a pLB zero-background rapid cloning kit, and transforming into Escherichia coli DH5α competent cells; conducting PCR amplification on an obtained selected bacterial plaque through a carrier primer, to obtain a PCR amplification product of the ITS sequence by monoclonal amplification. Further, an amplification system is 20 μL, including 10 μL of PCR 2× Mix, 10 μL of ddH₂O, 0.3 μL for each of the primers, and 1 cluster of a pLB-ligated bacterial plaque; the PCR amplification includes the following procedures: initial denaturation at 95° C. for 3 min; denaturation at 94° C. for 55 s, annealing at 55° C. for 55 s, and extension at 72° C. for 80 s, conducting 35 cycles; and extension at 72° C. for 7 min.

A sequencing reaction is required before the sequencing on a machine, the ITS and the matK have same reaction system and reaction conditions. A reaction system is 6 μL, including 0.3 μL of Bigdye, 1.0 μL of SeqBuffer, 3.70 μL of ddH₂O, 0.5 μL of a single-end primer, and 0.5 μL of a PCR purified product. The reaction conditions include: initial denaturation at 94° C. for 30 s; denaturation at 96° C. for 10 s, annealing at 50° C. for 5 s, and extension at 60° C. for 3 min, conducting 32 cycles. The sequencing is conducted with a Sanger sequencer, and sequence assembly is conducted on obtained sequencing results to obtain the matK sequence and the monoclonal ITS sequence for the parents to be determined of the Nymphaea.

After obtaining the ITS and matK sequences of the parents to be determined, sequence alignment is conducted with the standard database of the Nymphaea; a neighbor-joining tree is constructed based on a genetic distance; species sequences that are clustered with the parents to be determined are selected, the sequences are trimmed neatly, the sequence alignment and construction of the neighbor-joining tree are conducted for a second time, variation loci are checked to accurately determine a species closest to the parents to be determined; and the species is identified as a likely parental species.

Compared with the prior art, the present disclosure has the following advantages:

In the present disclosure, based on a large amount of published molecular sequence data of Nymphaea, an ITS sequence containing parental information and a matK sequence containing only maternal information are selected. After filtration, deduplication, alignment, and sorting, a database for each of the ITS and matK nucleotide sequences of Nymphaea is constructed. An applicable primer of the Nymphaea is designed to conduct PCR amplification; sequences of ITS and matK obtained by monoclonal sequencing are aligned with the respective database; a neighbor-joining tree is constructed based on a genetic distance, and specific loci are checked to obtain species information of male and female parents of the Nymphaea hybrid. The identification method is not only applicable to artificial hybrid cultivars, but also to naturally occurring hybrid species (conforming to ICBN regulations or conforming to the International Code for the Nomenclature for Cultivated Plants). By combining the ITS sequence of the nuclear genome (carrying the information of the two parents) and the matK sequence of the chloroplast genome (carrying the information of the female parent), the specific characteristics are obtained by sequence alignment and the N-J tree is constructed by the genetic distance, so as to obtain the species information of the male and female parents of the Nymphaea hybrid. The identification method can be widely used in the identification of cultivars of Nymphaea and the auxiliary verification of parental materials when breeding the Nymphaea.

### BRIEF DESCRIPTION OF THE DRAWINGS

In order to illustrate the specific embodiments of the present disclosure more clearly, the accompanying drawings required for the specific embodiments will be briefly introduced below.

FIG. 1 shows that 10 ITS sequences from monoclonal sequencing of Nymphaea ‘Joey Tomocik’ in Example 3 of the present disclosure, set forth in SEQ ID NOs: 6-14, are aligned with 50 sequences in an ITS database of the Nymphaea, and then a neighbor-joining tree is constructed, where a solid line box represents a clade gathered together with the 10 ITS sequences of the Nymphaea ‘Joey Tomocik’;

FIG. 2 shows that after the clade is selected from FIG. 1 in Example 3 of the present disclosure, the clade is aligned, a neighbor-joining tree is conducted, and a local sequence alignment diagram is displayed, where a dotted line box highlights Nymphaea mexicana and Nymphaea odorata subsp. tuberosa, and a solid line box shows molecular identification key points;

FIG. 3 shows alignment results of all ITS in Example 3 of the present disclosure, where a red solid line box shows key alignment sites for identification;

FIG. 4 shows that 2 matK sequences of Nymphaea ‘Joey Tomocik’ in Example 3 of the present disclosure, set forth in SEQ ID NOs: 15-16, are aligned with 32 sequences in a matK database of the Nymphaea, and then a neighbor-joining tree is constructed, where a solid line box represents a clade gathered together with the 2 matK sequences of the Nymphaea ‘Joey Tomocik’;

FIG. 5 shows that after clade is selected from FIG. 4 in Example 3 of the present disclosure, the clade is aligned, a neighbor-joining tree is conducted, and a local sequence alignment diagram is displayed, where a solid line box highlights specific loci of the matK.

### DETAILED DESCRIPTION OF THE EMBODIMENTS

The implementation of the present disclosure will be described below through specific examples. Unless otherwise stated, the experimental methods disclosed in the present disclosure all adopt conventional techniques in the technical field. The reagents and raw materials used in the examples are all commercially available. To help persons skilled in the art better understand the solutions of the present invention, the following clearly and completely describes the technical solutions in the embodiments of the present invention with reference to the accompanying drawings in the embodiments of the present invention.

### Example 1

This example provided a method for constructing a database for each of ITS and matK nucleotide sequences of Nymphaea. The method could update the constructed database of nucleotide sequences after addition of online public databases or newly-obtained sequences. The method specifically included the following steps:
1. Download of existing ITS and matK nucleotide sequences of Nymphaea. A nucleotide database of the National Center of Biotechnology Information (NCBI, www.ncbi.nlm.nih.gov/nucleotide) was searched with syntaxes “((Nymphaea[Organism]) AND 5.8S[Title]) NOT PREDICTED[Title]” and “((Nymphaea[Organism]) AND matK[Title]) NOT PREDICTED[Title]”, to obtain all data of the sequences of ITS and matK of the Nymphaea. A total of 499 ITS sequences were obtained, with a sequence length ranged from 213 bp to 1,002 bp. A total of 49 matK sequences were obtained, and 47 remained after removing two unverified sequences (KJ747597 and JQ024977). The 47 matK sequences had a length of 556 bp to 2,585 bp. In order to obtain more complete matK sequences of species, the NCBI was searched with a syntax “(chloroplast, complete genome[Title]OR plastid, complete genome[Title]) AND Nymphaea[Organism]” to obtain 59 published Nymphaea chloroplast genomes. The species with unclear identification were removed, and the remaining species were only taken from a RefSeq database, that is, 24 species beginning with “NC_”. The matK sequences of the chloroplast genomes of the 24 Nymphaea species were extracted.
2. Stringent filtration was conducted to retain one sequence for each “species unit”, namely species/subspecies/variety/form/hybrid of the sequences of ITS and matK. The alignment was conducted, the sequences with inconsistent sequence directions were adjusted, and then re-alignment was conducted to obtain the database of the nucleotide sequences ITS and matK of the Nymphaea. The stringent filtration specifically included:
a, a species whose species name included unverified, sp., and cf was removed, a subspecies (subsp.), a variety (var.), a form (f.), and a clear hybrid (x) that are included in the species were retained, which were referred to as “species units”; and each of an isolate, a voucher, a genotype, and a strain was regarded as a cultivar and the cultivar was classified into each “species unit”;
b, if the “species unit” had only one sequence, the filtration was omitted; after the filtration was conducted, only one sequence was retained for each “species unit”;
c, if a sequence of the cultivar in step a was quite different from that of the “species unit”, the sequence of the cultivar was removed preferentially;
d, a sequence with a significantly high difference (especially in a coding region such as 5.8S rRNA) was removed preferentially; a sequence having an unknown base and a degenerate base was removed preferentially; and a longer sequence was retained preferentially;
e, the matK sequence extracted from the chloroplast genomes was used only as a supplement; when the matK sequence obtained by sequencing already had the “species unit”, the matK sequence extracted from the chloroplast genomes was discarded preferentially; and
f, a sequence was retained as a sequence that best represented the “species unit” (a sequence being closest to a consensus sequence obtained from multiple sequences).
3. A sequence name of each obtained sequence was arranged, with a format of “species name|ACCESSION of sequence”. The ITS nucleotide database of Nymphaea included 50 sequences (Table 1), the matK nucleotide database of Nymphaea included 32 sequences (Table 2), and the sequences derived from the chloroplast genome were marked with “_plastome”.

TABLE 1
Sequence information of ITS nucleotide database of Nymphaea

| | Sequence length |
|---|---|
| Sequence name of ITS nucleotide database of Nymphaea | (bp) |
| Nymphaea_alba_var._rubra_clone_Nyarclo10\|GU222355 | 663 |
| Nymphaea_alba\|HG518071 | 684 |
| Nymphaea_amazonum\|FR717598 | 746 |
| Nymphaea_ampla_strain_100N\|AY771812 | 643 |
| Nymphaea_atrans_isolate_NY432\|FJ026555 | 797 |
| Nymphaea_caerulea_isolate_NycW3\|GQ468651 | 801 |
| Nymphaea_candida\|HG518075 | 684 |
| Nymphaea_capensis\|AY620421 | 704 |
| Nymphaea_colorata_x_Nymphaea_gigantea_genotype_2x\|AY390336 | 644 |
| Nymphaea_colorata\|HF968668 | 684 |
| Nymphaea_elleniae_isolate_NY103\|FJ026560 | 797 |
| Nymphaea_georginae_isolate_NY425\|FJ026563 | 720 |
| Nymphaea_gigantea_genotype_4x\|AY390327 | 657 |
| Nymphaea_gracilis\|FR717586 | 690 |
| Nymphaea_guineensis\|FR717590 | 772 |
| Nymphaea_hastifolia_isolate_NY134\|FJ026568 | 780 |
| Nymphaea_heudelotii_isolate_NY066\|FJ026603 | 739 |
| Nymphaea_immutabilis_isolate_NY121\|FJ026569 | 747 |
| Nymphaea_immutabilis_subsp._kimberleyensis_isolate_NY380\|FJ026576 | 287 |
| Nymphaea_jamesoniana\|FR717597 | 697 |
| Nymphaea_leibergii_x_Nymphaea_odorata_3328b\|HG518096 | 628 |
| Nymphaea_leibergii\|HG518083 | 688 |
| Nymphaea_lingulata\|HG518070 | 705 |
| Nymphaea_lotus_f._thermalis\|FM242153 | 677 |
| Nymphaea_lotus_var._dentata\|HF968670 | 675 |
| Nymphaea_lotus_voucher_MSoLS_NL_1\|OM677814 | 746 |
| Nymphaea_macrosperma_isolate_NY127\|FJ026579 | 794 |
| Nymphaea_mexicana_isolate_Knysna_estate_1\|OM614904 | 723 |
| Nymphaea_micrantha\|FR717593 | 764 |
| Nymphaea_minuta_voucher_HELLQ\|EU428066 | 706 |
| Nymphaea_nouchali_isolate_G2\|FJ597742 | 695 |
| Nymphaea_nouchali_var._caerulea_isolate_671\|MW798802 | 703 |
| Nymphaea_novogranatensis_isolate_NY-021\|FM242154 | 490 |
| Nymphaea_odorata_subsp._odorata_voucher_DRAGON_1\|EU428067 | 672 |
| Nymphaea_odorata_subsp._tuberosa\|HG518107 | 686 |
| Nymphaea_odorata_x_Nymphaea_tetragona_16163b\|HG518082 | 621 |
| Nymphaea_odorata\|AY858641 | 731 |
| Nymphaea_ondinea_isolate_NY377\|FJ026600 | 730 |
| Nymphaea_oxypetala_isolate_NY-387\|FM242150 | 682 |
| Nymphaea_petersiana_isolate_GMN\|MW798869 | 691 |
| Nymphaea_pubescens_isolate_NPUBITS\|FJ198406 | 680 |
| Nymphaea_pulchella_isolate_NY567\|FR717596 | 700 |
| Nymphaea_pygmaea\|HG518076 | 699 |
| Nymphaea_rubra\|GU199468 | 680 |
| Nymphaea_rudgeana\|HG518069 | 707 |
| Nymphaea_tetragona_var._wenzelii_voucher_738-03\|EU428054 | 680 |
| Nymphaea_tetragona_voucher_51\|EU428031 | 680 |
| Nymphaea_violacea_isolate_NY413\|FJ026597 | 767 |
| Nymphaea_x_marliacea_isolate_NMARITS\|FJ198403 | 662 |
| Nymphaea_zenkeri_isolate_CAM01\|MW798844 | 690 |

TABLE 2
Sequence information of matK nucleotide database of Nymphaea

| Sequence name of matK | Sequence length |
|---|---|
| nucleotide database of Nymphaea | (bp) |
| Nymphaea_alba\|HE967445 | 729 |
| Nymphaea_amazonum\|DQ185543 | 2868 |
| Nymphaea_atrans_plastome\|NC_060672 | 1530 |
| Nymphaea_caerulea_isolate_NycW1\|GQ468658 | 2571 |
| Nymphaea_colorata_plastome\|NC_057562 | 1530 |
| Nymphaea_conardii_plastome\|NC_056819 | 1515 |
| Nymphaea_elleniae\|DQ185539 | 2801 |
| Nymphaea_gigantea_plastome\|NC_057568 | 1542 |
| Nymphaea_glandulifera_plastome\|NC_056820 | 1515 |
| Nymphaea_gracilis\|DQ185542 | 2831 |
| Nymphaea_heudelotii_plastome\|NC_056822 | 1530 |
| Nymphaea_immutabilis_plastome\|NC_056823 | 1530 |
| Nymphaea_jamesoniana\|DQ185544 | 2849 |
| Nymphaea_lotus\|DQ185547 | 2826 |
| Nymphaea_macrosperma\|DQ185540 | 2912 |
| Nymphaea_mexicana_isolate_HVK0026d\|KJ747596 | 736 |
| Nymphaea_micrantha\|DQ185541 | 2846 |
| Nymphaea_nouchali_isolate_C1\|FJ597752 | 2532 |
| Nymphaea_nouchali_var._caerulea_isolate_ | 659 |
| HVK007b\|KJ747541 | |
| Nymphaea_novogranatensis\|DQ185545 | 2872 |
| Nymphaea_odorata\|DQ185549 | 2874 |
| Nymphaea_ondinea\|DQ185538 | 2864 |
| Nymphaea_oxypetala\|DQ185546 | 2891 |
| Nymphaea_petersiana\|DQ185548 | 2840 |
| Nymphaea_potamophila_plastome\|NC_057564 | 1515 |
| Nymphaea_pubescens_isolate_H1\|FJ597753 | 2519 |
| Nymphaea_rubra_isolate_D1\|FJ597754 | 2519 |
| Nymphaea_rudgeana_plastome\|NC_056824 | 1524 |
| Nymphaea_tenerinervia_plastome\|NC_056825 | 1515 |
| Nymphaea_tetragona_isolate_E1\|FJ597755 | 2518 |
| Nymphaea_thermarum_plastome\|NC_056953 | 1530 |
| Nymphaea_x_marliacea_isolate_NymM1\|GQ358630 | 2530 |

### Example 2

This example provided a method for obtaining an ITS (monoclonal) nucleotide sequence and a matK nucleotide sequence of the species for the parents to be determined of Nymphaea, including the following steps:
1. The genomic DNA of the Nymphaea of the parents to be determined was extracted. The total DNA from Nymphaea leaves was extracted using a modified 2×CTAB method (ROGERS and BENDICH, 1985). This method included grinding under liquid nitrogen, treating with a CTAB extracting solution in a 65° C. water bath, extraction with chloroform and isoamyl alcohol, sedimentation with isoamyl alcohol, ethanol washing, and ribonuclease digestion.
2. The primers were designed. The 18S and 25S sequence data of Nymphaea were downloaded from NCBI's nucleotide database. Combined with the ITS sequence data downloaded in Example 1, the commonly used ITS primer pairs in plants were searched. In this way, the corresponding primers with appropriate product size and high complementarity to Nymphaea 18S and 25S sequences were found as a primer pair for the PCR amplification of Nymphaea ITS. The ITS primer pair included: a forward primer ITS-5 (AGTCGTAACAAGGTTTCCGT) (SEQ ID NO: 1) and a reverse primer ITS-3 (TAGTAACGGCGAGCGAACC) (SEQ ID NO: 2. Similarly, a matK primer pair commonly used in plants were searched and aligned with the matK sequences downloaded in Example 1, and inconsistent bases were modified to make the primer pair highly complementary to the matK sequences of Nymphaea. The matK primer pair included: a forward primer matK-5 (CGTACCGTACTTTTATGTTTACGAG) (SEQ ID NO: 3) and a reverse primer matK-3 (ACCCAATCCATCTGGAAATCTTGCTTC) (SEQ ID NO: 4).
3. PCR sequence amplification. A PCR amplification product of the ITS was obtained by the primer pair ITS-5/ITS-3, and a PCR amplification product of the matK was obtained by the primer pair matK-5/matK-3. A PCR amplification system of the ITS sequence was 30 μL, including 15 μL of PCR 2× Mix, 13 μL of ddH₂O, 0.5 μL for each of the primers, and 1.0 μL of a total DNA. The PCR amplification of the ITS sequence included the following procedures: initial denaturation at 95° C. for 3 min; denaturation at 94° C. for 55 s, annealing at 55° C. for 55 s, and extension at 72° C. for 70 s, conducting 35 cyles; and extension at 72° C. for 7 min. A PCR amplification system of the matK sequence was 20 μL, including 10 μL of the PCR 2× Mix, 8 μL of the ddH₂O, 0.5 μL for each of the primers, and 1.0 μL of the total DNA. The PCR amplification of the matK sequence included the following procedures: initial denaturation at 95° C. for 3 min; denaturation at 94° C. for 30 s, annealing at 52° C. for 30 s, and extension at 72° C. for 1 min, conducting 35 cyles; and extension at 72° C. for 5 min. The obtained PCR amplification products were detected by 1% agarose gel electrophoresis (4S green dye, 150 V, 25 min of electrophoresis), and the lengths of the amplification products were about 900 bp (ITS) and 1,000 bp (matK). The PCR products were purified with a SANPREP column PCR product purification kit, and agarose gel electrophoresis was conducted to detect whether the PCR products were not eluted. After the PCR amplification products of ITS were obtained with the primer pair ITS-5/ITS-3, a cohesive end was ligated using a 5 μL system of a pLB zero-background rapid cloning kit (Tiangen CT205). 5 μL of an obtained ligation product was added to DH5α competent cells (Tiangen CB101), transformed, spread evenly on a solid LB medium (containing ampicillin), and incubated at 37° C. for 15 h. A bacterial plaque was selected with a pipette tip and washed into a PCR amplification system of a carrier primer. An amplification system was 20 μL, including 10 μL of PCR 2× Mix, 10 μL of ddH₂O, 0.3 μL for each of the primers, and 1 cluster of a pLB-ligated bacterial plaque. The PCR amplification included the following procedures: initial denaturation at 95° C. for 3 min; denaturation at 94° C. for 55 s, annealing at 55° C. for 55 s, and extension at 72° C. for 80 s, conducting 35 cycles; and extension at 72° C. for 7 min. The PCR amplification products of the carrier primer were detected by the 1% agarose gel electrophoresis.
4. Sanger sequencing. Before sequencing on a machine, PCR product purification and sequencing reaction were required. The PCR product purification was conducted using a purification enzyme (5 μL PCR product: 2 μL PCR purification enzyme E), at 37° C. and 80° C. separately for 15 min. The ITS and the matK had same reaction system and reaction conditions during the sequencing reaction. A reaction system was 6 μL, including 0.3 μL of Bigdye, 1.0 μL of SeqBuffer, 3.70 μL of ddH₂O, 0.5 μL of a single-end primer, and 0.5 μL of a PCR purified product. The reaction conditions included: initial denaturation at 94° C. for 30 s; denaturation at 96° C. for 10 s, annealing at 50° C. for 5 s, and extension at 60° C. for 3 min, conducting 32 cycles. The product of the sequencing reaction was then settled with a sedimentation agent (95% ethanol: sodium acetate at 20:1), and eluted with 75% ethanol to obtain a purified sequencing reaction product. The purified product was sequenced on a 3730XL sequencer after denaturation, and sequence assembly was conducted using a software on obtained sequencing results to obtain the matK sequence and the monoclonal ITS sequence for the parents to be determined of the Nymphaea.
5. Sequence alignment and construction of neighbor-joining tree. After obtaining the ITS and matK sequences of the parents to be determined, sequence alignment was conducted with the standard database of the Nymphaea; a neighbor-joining tree was constructed based on a genetic distance (the alignment and the N-J tree construction were conducted using Geneious software). The species sequences that were clustered with the parents to be determined were selected, the sequences were trimmed neatly, the sequence alignment and construction of the neighbor-joining tree were conducted for a second time, variation loci were checked to accurately determine a species closest to the parents to be determined; and the species was identified as a likely parental species.

### Example 3

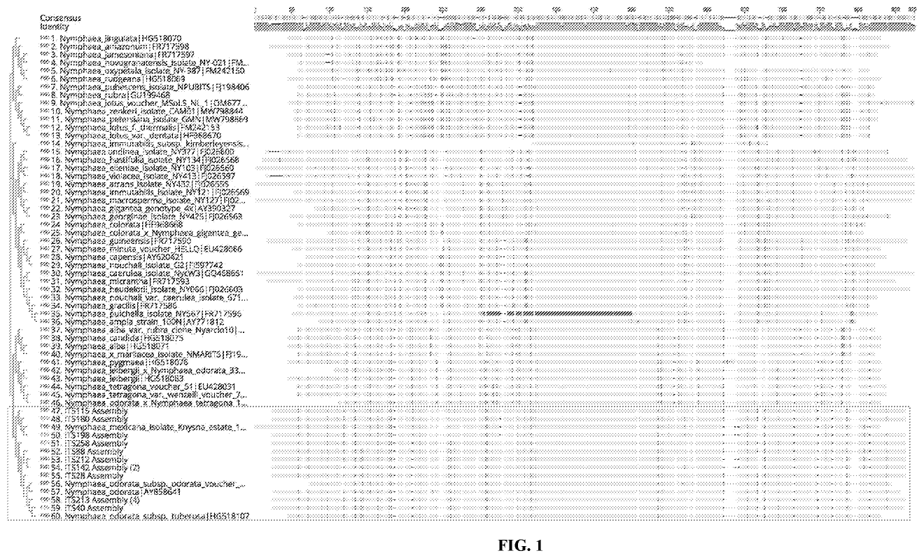

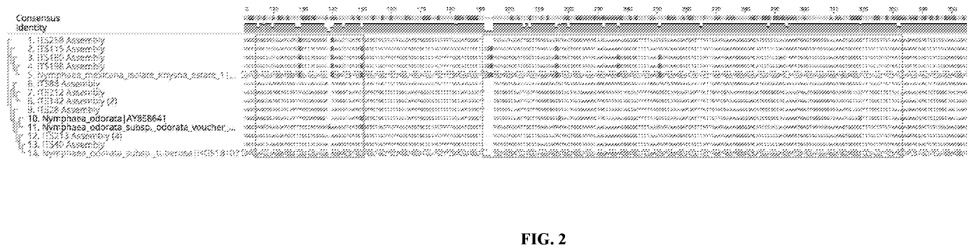

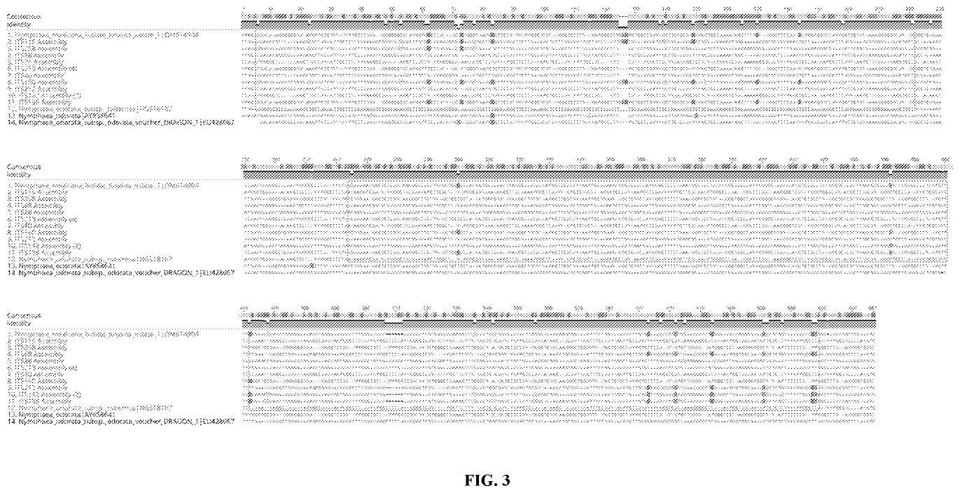

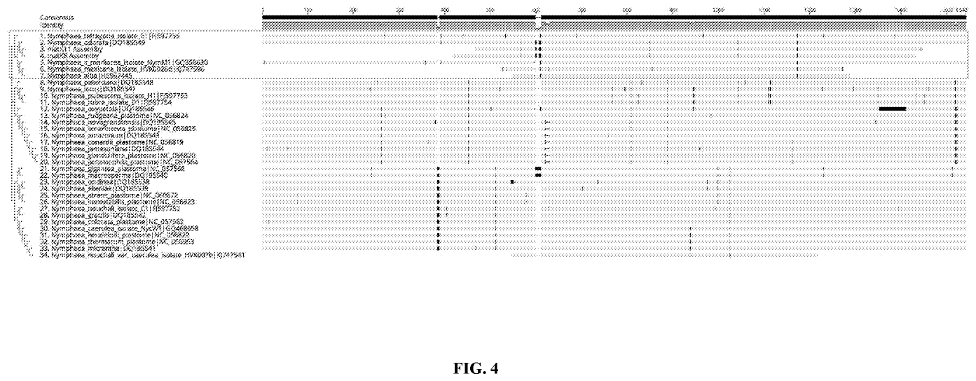

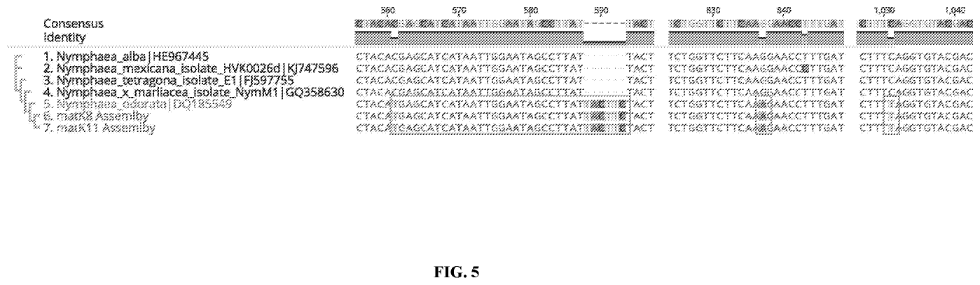

The leaves of a Nymphaea variety N. ‘Joey Tomocik’ planted in the botanical garden of Kunming Institute of Botany, Chinese Academy of Sciences, were used as materials for DNA extraction, PCR amplification, and sequencing using the method in Example 2. A total of 10 sequences of 10 “genotypes” were obtained from a monoclonal ITS sequence of this variety, and a sequence lengths was 855 bp to 935 bp (FIG. 1, FIG. 2, and FIG. 3); 2 matK sequences of 1 genotype were obtained, and the sequence lengths were 977 bp and 1,008 bp, respectively (FIG. 4 and FIG. 5).

The 10 monoclonal ITS sequences of N. ‘Joey Tomocik’ were aligned with the database of ITS nucleotide sequences of Nymphaea constructed in Example 1, and a neighbor-joining tree was constructed. The results showed that the 10 ITS sequences of N. ‘Joey Tomocik’ and N. mexican and N. odorata (including its two varieties subsp. odorata and subsp. tuberosa) were clustered into a clade (FIG. 1). A sequence of the clade was extracted and then aligned to construct a neighbor-joining tree. The results showed that the topology of the tree remained unchanged, where 4 sequences were clustered with the N. mexican and another 6 sequences were clustered with the N. odorata. After changing a view of the neighbor-joining tree to a sequence view, it was seen that most of the specific loci of ITS115, ITS180, and ITS198 came from the N. mexican, while most of the specific loci of ITS88, ITS212, ITS142, ITS28, ITS213, and ITS40 came from the N. odorata, specifically a variety (N. odorata subsp. tuberosa).). This was because both N. odorata and the original variety had loci that N. ‘Joey Tomocik’ did not contain. ITS258 were balanced in the N. mexican and N. odorata (FIG. 2). By carefully aligning all the sequences, it was found that the specific loci of the 10 monoclonal ITS sequences sequenced in N. ‘Joey Tomocik’ and those in the N. mexican and N. odorata intersected with each other (FIG. 3). This showed that the two parents of the N. ‘Joey Tomocik’ should be N. mexican and N. odorata subsp. tuberosa, but the male parent and the female parent could not be determined yet.

The 2 matK sequences of N. ‘Joey Tomocik’ were aligned with the database of matK nucleotide sequences of Nymphaea constructed in Example 1, and a neighbor-joining tree was constructed. The results showed that the matK sequences of the N. ‘Joey Tomocik’ were clustered into a clade with N. mexican, N. odorata, N. tetragona, N. alba, and a hybrid N. x marliacea (FIG. 4). A sequence of the clade was extracted and then aligned to construct a neighbor-joining tree. The results showed that the 2 matK sequences of N. ‘Joey Tomocik’ and N. odorata belonged to one clade. After changing a neighbor-joining tree view to sequence view, it was seen that several specific loci of the N. ‘Joey Tomocik’ were completely consistent with the N. odorata, but not with the other three variety. This showed that the female parent of N. ‘Joey Tomocik’ should be N. odorata. Combined with the identification results of the previous ITS sequences, it was concluded that the hybrid parents of N. ‘Joey Tomocik’ were: male parent N. mexican and female parent N. odorata. As for whether the female parent should be the variety of N. odorata, N. odorata subsp. tuberosa, the final determination could only be made after the matK nucleotide database of Nymphaea was updated until there were two varieties of the N. odorata. The results identified in this example were consistent with the parents of an ambiguous record of the N. ‘Joey Tomocik’ in the literature (POWELL, 2009)—“parented by N. mexicana on an odorata rhizome”. This had also verified the accuracy of the present disclosure from the side.

The above are merely preferred implementations of the present disclosure. It should be noted that several improvements and modifications may further be made by a person of ordinary skill in the art without departing from the principle of the present disclosure, and such improvements and modifications should also be deemed as falling within the protection scope of the present disclosure.

```
Sequence Listing Information:
  DTD Version: V1_3
  File Name: GWP20230402643.xml
  Software Name: WIPO Sequence
  Software Version: 2.1.2
  Production Date: 2023 May 16
General Information:
  Current application/Applicant file reference: GWP20230402643
  Earliest priority application/IP Office: CN
  Earliest priority application/Application number: 202210533264.8
  Earliest priority application/Filing date: 2022 May 17
  Applicant name: Kunming Institute of Botany, CAS
  Applicant name/Language: en
  Invention title: IDENTIFICATION METHOD OF TWO PARENTS OF NYMPHAEA
HYBRID BASED ON SEQUENCES OF INTERNAL TRANSCRIBED SPACER (ITS) AND
matK (en)
  Sequence Total Quantity: 16
Sequences:
Sequence Number (ID): 1
  Length: 20
  Molecule Type: DNA
  Features Location/Qualifiers:
    - source, 1 .. 20
      > mol_type, other DNA
      > note, Primer ITS-5
      > organism, synthetic construct
  Residues:
  agtcgtaaca aggtttccgt 20
  Sequence Number (ID): 2
  Length: 19
  Molecule Type: DNA
  Features Location/Qualifiers:
    - source, 1 .. 19
      > mol_type, other DNA
      > note, Primer ITS-3
      > organism, synthetic construct
  Residues:
  tagtaacggc gagcgaacc
  19
  Sequence Number (ID): 3
  Length: 25
  Molecule Type: DNA
  Features Location/Qualifiers:
    - source, 1 .. 25
      > mol_type, other DNA
      > note, Primer matK-5
      > organism, synthetic construct
  Residues:
  cgtaccgtac ttttatgttt acgag 25
  Sequence Number (ID): 4
  Length: 27
  Molecule Type: DNA
  Features Location/Qualifiers:
    - source, 1 .. 27
      > mol_type, other DNA
      > note, Primer matK-3
      > organism, synthetic construct
  Residues:
  acccaatcca tctggaaatc ttgcttc 27
  Sequence Number (ID): 5
  Length: 860
  Molecule Type: DNA
  Features Location/Qualifiers:
    - source, 1 .. 860
      > mol_type, genomic DNA
      > note, ITS28_Assembly
      > organism, Nymphaea ‘Joey Tomocik’
  Residues:
  tagggagagc ggccgccgga tctcccggat ggttcgagtt tttcagcaag atgtcgtaac 60
  naaggtttcc gtaggtgaac ctgcggaagg atcattgtcg tttcctttta gatgacagac 120
  ccgcgaacaa gttatcattg ctttcttcaa gccgagcgga gcatcgtttc ccacgggaaa 180
  gggtgatctg ttctgcttct tggcatgtgc ccgtcttttg ccatcccctt tgtggtcgat 240
  tgcttgagcg gtgtactgcc aaaacaacaa aaacggcgct tttaagtgtc aaggatcatt 300
  tattgaatga aagggggaca tcccgccaca aaatgagtgg ggggagaagt gcccctttgc 360
  cttctaacaa gaacgactct cggcaacgga tatcttggct cctgtcacga tgaagaacgt 420
  agcgaaatgc gatagttggt gtgaattgca gaatcccgtg aatcatcgag tttttgaacg 480
  caagttgcgc ccgaggccat tcggccaagg gcacgtctgc ctgggcgtca cgcttagcgt 540
  cgccctcccc aggtcctcgt gttttcgaac cgaggccgag ggagagcgga ggactggcct 600
  tcggtgtcgc tttcatcggc gtcgtcggct gaaactttcg gctcacgatc tgttgtgcag 660
  cacaacaagc ggtggatttc cagtgagttg ttgtgtttca cgtggtcgaa gggccatggg 720
  actcgaggca aggttctcat tttcttgcct tagctttgcg accccaggtc aggcgagact 780
  acccgctgag tttaagcata tcaataagcg gatctctcta gcaggtctcc tacaatattc 840
  tcagctgcca tggaaaatcg 860
  Sequence Number (ID): 6
  Length: 857
  Molecule Type: DNA
  Features Location/Qualifiers:
    - source, 1 .. 857
      > mol_type, genomic DNA
      > note, ITS40_Assembly
      > organism, Nymphaea sp.
  Residues:
  ttcgattttc catggcagct gagaatattg taggagactg ctagagagat agtcgtaaca 60
  atttccgtag gtgaacctgc ggaaggatca ttgtcgtttc cttagatgac agacccgcga 120
  acaagttatc attgctttct tcaagccgag cggagcatcg tttcccacgg gaaagggtga 180
  tctgttctgc ttcttggcat gtgcccgtct tttgccatcc cctttgtggt cgattgcttg 240
  agcggtgtac tgccaaaaca acaaaaacgg cgcttttaag tgtcaaggat catttattga 300
  atgaaagggg gacatcccgc cacaaaatga gtggggggag aagtgcccct ttgccttcta 360
  acaagaacga ctctcggcaa cggatatctt ggctcctgtc acgatgaaga acgtagcgaa 420
  atgcgatagt tggtgtgaat tgcagaatcc cgtgaatcat cgagtttttg aacgcaagtt 480
  gcgcccgagg ccattcggcc aagggcacgt ctgcctgggc gtcacgctta gcgtcgccct 540
  ccccaggtcc tcgtgttttc gaaccgaggc cgagggagag cggaggactg gccttcggtg 600
  tcgctttcat cggcgtcgtc ggctgaaact ttcggctcac gatctgttgt gcagcacaac 660
  aagcggtgga tttccagtga gttgttgtgt ttcacgtggt caaagggcca tgggactcga 720
  ggcaaggttc tcattttctt gccttagctt tgcgacccca ggtcaggcga gactacccgc 780
  tgagtttaag catatctngc tgaaaaactc gaaccatccc gggagatccc ggggccgct 840
  ctccctatag gtgagtc 857
  Sequence Number (ID): 7
  Length: 860
  Molecule Type: DNA
  Features Location/Qualifiers:
    - source, 1 .. 860
      > mol_type, genomic DNA
      > note, ITS88_Assembly
      > organism, Nymphaea sp.'
  Residues:
  ttttccatgg cagctgagaa tatgtaggag acctgctaga gagatagtcg taacaaggtt 60
  tccgtaggtg aacctgcgga aggatcattg tcgtttcctt ttagatgaca gacccgcgaa 120
  caagttatca ttgctttctt caagccgagc ggagcatcgt ttcccacggg aaagggtgat 180
  ctgttctgct tcttggcatg tgcccgtctt ttgccatccc ctttgtggtc gattgcttga 240
  gcggtgtact gccaaaacaa caaaaacggc gcttttaagt gtcaaggatc atttattgaa 300
  tgaaaggggg acatcccgcc acaaaatgag tggggggaga agtgcccctt tgccttctaa 360
  caagaacgac tctcggcaac ggatatcttg gctcctgtca cgatgaagaa cgtagcgaaa 420
  tgcgatagtt ggtgtgaatt gcagaatccc gtgaatcatc gagtttttga acgcaagttg 480
  cgcccgaggc cattcggcca agggcacgtc tgcctgggcg tcacgcttag cgtcgccctc 540
  cccaggtcct cgtgttttcg aaccgaggcc gagggagagc ggaggactgg ccttcggtgt 600
  cgctttcatc ggcgtcgtcg gctgaaactt tcggctcacg atctgttgtg cagcacaaca 660
  agcggtggat ttccagtgag ttgttgtgct tcgcgtgatc gaagggccac gggactcgag 720
  gcaaggagct cagtttcttg cccaagcttt gcgaccccag gtcaggcgag actacccgct 780
  gagtttaagc atatcaataa gcggaggaat cttgctgaaa aactcgaacc atccgggaga 840
  tccggcggcc gctctcccta 860
  Sequence Number (ID): 8
  Length: 855
  Molecule Type: DNA
  Features Location/Qualifiers:
    - source, 1 .. 855
      > mol_type, genomic DNA
      > note, ITS115_Assembly
      > organism, Nymphaea sp.
  Residues:
  ttccatggca gctgagaata ttgtaggaga ctgctagaga gatagtcgta acaatttccg 60
  taggtgaacc tgcggaagga tcattgtcgt ttccttagag gacagacccg cgaacatgtt 120
  atcattgctt tcttcaagcc gggcggtgca tcgtgcccca cggggcaggt tgatccgttc 180
  tgcttcttgg catgtgcccg tcttttgcca tcccctttac tgtggtcgat ggtttgagcg 240
  gcgtattgcc aaaacaataa aaacggcgct tttaagtgtc aaggatcatt tattgaatga 300
  aagggggaca tcccgccaca aaatgagtgg ggggagaagt gcccctttgc cttctaacaa 360
  gaacgactct cggcaacgga tatcttggct cctgtcacga tgaagaacgt agcgaaatgc 420
  gatagttggt gtgaattgca gaatcccgtg aatcatcgag tttttgaacg caagttgcgc 480
  ccgaggccat tcggccaagg gcacgtctgc ctgggcgtca cgcttagcgt cgccctcccc 540
  aggtcctcgt gttttcgaac cgaggccgag ggagagcgga ggactggcct tcggtgtcgc 600
  tttcatcggc gtcgtcggct gaaactttcg gctcacgatc tgttgtgcag cacaacaagc 660
  ggtggatttc cagtgagttg ttgtgtttca cgtggtcaaa gggccatggg actcgaggca 720
  aggttctcat tttcttgcct tagctttgcg accccaggtc aggcgagact acccgctgag 780
  tttaagcata tcaataagcg gaggatcttg ctgaaaaact cgaaccatcc gggagatccg 840
  gcggccgctc tccta 855
  Sequence Number (ID): 9
  Length: 856
  Molecule Type: DNA
  Features Location/Qualifiers:
    - source, 1 .. 856
      > mol_type, genomic DNA
      > note, ITS142_Assembly
      > organism, Nymphaea sp.
  Residues:
  tagggagagc ggccgccgga tctcccggat ggttcgagtt tttcagcaag atagtcgtaa 60
  caatttccgt aggtgaacct gcggaaggat cattgtcgtt tccttagatg acagacccgc 120
  gaacaagtta tcattgcttt cttcaagccg agcggagcat cgtttcccac gggaaagggt 180
  gatctgttct gcttcttggc atgtgcccgt cttttgccat cccctttgtg gtcgattgct 240
  tgagcggtgt actgccaaaa caacaaaaac ggcgctttta agtgtcaagg atcatttatt 300
  gaatgaaagg gggacatccc gccacaaaat gagtgggggg agaagtgccc ctttgccttc 360
  taacaagaac gactctcggc aacggatatc ttggctcctg tcacgatgaa gaacgtagcg 420
  aaatgcgata gttggtgtga attgcagaat cccgtgaatc atcgagtttt tgaacgcaag 480
  ttgcgcccga ggccattcgg ccaagggcac gtctgcctgg gcgtcacgct tagcgtcgcc 540
  atccccaggt cctcgtgttt ccgaactgag gccgagggag agcggaggac tggccttcgg 600
  tgtcgtcggc gtcgtcggct gaaacttttg gctcgcgatc tgttgtgcgg cacaacaagc 660
  ggtggatttc cagtgagttg ttgtgcttcg cgtgatcgaa gggccacggg actcgaggca 720
  aggagctcag tttcttgccc aagctttgcg accccaggtc aggcgagact acccgctgag 780
  tttaagcata tcaataagcg gaggaatctc tctagcaggt ctcctacaat attctcagct 840
  gccatggaaa aatcga 856
  Sequence Number (ID): 10
  Length: 886
  Molecule Type: DNA
  Features Location/Qualifiers:
    - source, 1 .. 886
      > mol_type, genomic DNA
      > note, ITS180_Assembly
      > organism, Nymphaea sp.
  Residues:
  tcaaaaaaca tcgattttcc atggcagctg agaatattgt aggagacctg ctagagagat 60
  agtcgtaaca atttccgtag gtgaacctgc ggaaggatca ttgtcgtttc cttagaggac 120
  agacccgcga acatgttatc attgctttct tcaagccggg cggtgcatcg tgccccacgg 180
  ggcaggttga tccgttctgc ttcttggcat gtgcccgtct tttgccatcc cctttactgt 240
  ggtcgatggt ttgagcggcg tattgccaaa acaataaaac cggcgctttt aagcgtcaag 300
  gatcattgat tgaatgaaag ggggacatcc tgccacaaat gagtgggggg agaagtgccc 360
  ctttgccttc taataagaac gactctcggc aacggatatc ttggctcccg tcacgatgaa 420
  gaacgtagcg aaatgcgata gttggtgtga attgcagaat cccgtgaatc atcgagtttt 480
  tgaacgcaag ttgcgcccga ggccattcgg ccaagggcac gtctgcctgg gcgtcacgct 540
  tagcgtcgcc atccccaggt cctcgtgttt ccgaactgag gccgagggag agcggaggac 600
  tggccttcgg tgtcgctttc atcggcgtcg tcggctgaaa ctttcggctc acgatctgtt 660
  gtgcagcaca acaagcggtg gatttccagt gagttgttgt gtttcacgtg gtcaaagggc 720
  catgggactc gaggcaaggt tctcattttc ttgccttagc tttgcgaccc caggtcaggc 780
  gagactaccc gctgagttta agcatatcaa taagcggaga tcttgctgaa aaactcgaac 840
  catccmgkag atcccggcgg ccgctctccc tataggtgga gtcaga 886
  Sequence Number (ID): 11
  Length: 866
  Molecule Type: DNA
  Features Location/Qualifiers:
    - source, 1 .. 866
      > mol_type, genomic DNA
      > note, ITS198_Assembly
      > organism, Nymphaea sp.
  Residues:
  aaaggaacac actgcgtttc catggcagct gagaatattg taggagactg ctagagagat 60
  agtcgtaaca atttccgtag gtgaacctgc ggaaggatca ttgtcgtttc cttagaggac 120
  agacccgcga acatgttatc attgctttct tcaagccggg cggtgcatcg tgccccacgg 180
  ggcaggttga tccgttctgc ttcttggcat gtgcccgtct tttgccatcc cctttactgt 240
  ggtcgatggt ttgagcggcg tattgccaaa acaataaaac cggcgctttt aagcgtcaag 300
  gatcattgat tgaatgaaag ggggacatcc tgccacaaat gagtgggggg agaagtgccc 360
  ctttgccttc taataagaac gactctcggc aacggatatc ttggctcccg tcacgatgaa 420
  gaacgtagcg aaatgcgata gttggtgtga attgcagaat cccgtgaatc atcgagtttt 480
  tgaacgcaag ttgcgcccga ggccattcgg ccaagggcac gtctgcctgg gcgtcacgct 540
  tagcgtcgcc atccccaggt cctcgtgttt ccgaattgag gccgagggag agcggaggac 600
  tggccttcgg tgtcgtcggc gtcgtcggct gaaacttttg gctcgcgatc tgttgtgcgg 660
  cacaacaagc ggtggatttc cagtgagttg ttgtgcttcg cgtgatcgaa gggccacggg 720
  actcgaggca aggagctcag tttcttgccc aagctttgcg accccaggtc aggcgagact 780
  acccgctgag tttaagcata tcaataagcg gaggatcttg ctgaaaaact cgaaccatcc 840
  gggagatccg gcggccgctc tccttt 866
  Sequence Number (ID): 12
  Length: 881
  Molecule Type: DNA
  Features Location/Qualifiers:
    - source, 1 .. 881
      > mol_type, genomic DNA
      > note, ITS212_Assembly
      > organism, Nymphaea sp.
  Residues:
  tcgactcacc tatagggaga gcggccgccg gatctcccgg atggttcgag tttttcagca 60
  agatagtcgt aacaatttcc gtaggtgaac ctgcggaagg atcattgtcg tttccttaga 120
  tgacagaccc gcgaacaagt tatcattgct ttcttcaagc cgagcggagc atcgtttccc 180
  acgggaaagg gtgatctgtt ctgcttcttg gcatgtgccc gtcttttgcc atcccctttg 240
  tggtcgattg cttgagcggt gtactgccaa aacaacaaaa acggcgcttt taagtgtcaa 300
  ggatcattta ttgaatgaaa gggggacatc ccgccacaaa atgagtgggg ggagaagtgc 360
  ccctttgcct tctaacaaga acgactctcg gcaacggata tcttggctcc tgtcacgatg 420
  aagaacgtag cgaaatgcga tagttggtgt gaattgcaga atcccgtgaa tcatcgagtt 480
  tttgaacgca agttgcgccc gaggccattc ggccaagggc acgtctgcct gggcgtcacg 540
  cttagcgtcg ccctccccag gtcctcgtgt tttcgaaccg aggccgaggg agagcggagg 600
  actggccttc ggtgtcgtcg gcgtcgtcgg ctgaaacttt tggctcgcga tctgttgtgc 660
  ggcacaacaa gcggtggatt tccagtgagt tgttgtgctt cgcgtgatcg aagggccacg 720
  ggactcgagg caaggagctc agtttcttgc ccaagctttg cgaccccagg tcaggcgaga 780
  ctacccgctg agtttaagca tatcaataag cggaggaatc tctctagcag tctcctacaa 840
  tattctcagc tgccatggaa atcgaaatgg tttctttata a 881
  Sequence Number (ID): 13
  Length: 936
  Molecule Type: DNA
  Features Location/Qualifiers:
    - source, 1 .. 936
      > mol_type, genomic DNA
      > note, ITS213_Assembly
      > organism, Nymphaea sp.'
  Residues:
  ctaaagagaa cactattgcg atttccatgg cagctgagaa tatgtaggag actgctagag 60
  agatagtcgt aacaaggttt ccgtaggtga acctgcggaa ggatcattgt cgtttccttt 120
  tagatgacag acccgcgaac aagttatcat tgctttcttc aagccgagcg gagcatcgtt 180
  tcccacggga aagggtgatc tgttctgctt cttggcatgt gcccgtcttt tgccatcccc 240
  tttgtggtcg attgcttgag cggtgtactg ccaaaacaac aaaaacggcg cttttaagtg 300
  tcaaggatca tttattgaat gaaaggggga catcccgcca caaaatgagt ggggggagaa 360
  gtgccccttt gccttctaac aagaacgact ctcggcaacg gatatcttgg ctcctgtcac 420
  gatgaagaac gtagcgaaat gcgatagttg gtgtgaattg cagaatcccg tgaatcatcg 480
  agtttttgaa cgcaagttgc gcccgaggcc attcggccaa gggcacgtct gcctgggcgt 540
  cacgcttagc gtcgccctcc ccaggtcctc gtgttttcga accgaggccg agggagagcg 600
  gaggactggc cttcggtgtc gctttcatcg gcgtcgtcgg ctgaaacttt cggctcacga 660
  tctgttgtgc agcacaacaa gcggtggatt tccagtgagt tgttgtgttt cacgtggtca 720
  aagggccatg ggactcgagg caaggttctc attttcttgc cttagctttg cgaccccagg 780
  tcaggcgaga ctacccgctg agtttaagca tatcaataag cggaggaaaa gaaacttaca 840
  aggattcccc tagtaacggc gagcgaacca tcttgctgaa aaactcgaac catcccggga 900
  gatcccggcg gccgctctcc ctataggtga gtcgaa 936
  Sequence Number (ID): 14
  Length: 889
  Molecule Type: DNA
  Features Location/Qualifiers:
    - source, 1 .. 889
      > mol_type, genomic DNA
      > note, ITS258_Assembly
      > organism, Nymphaea sp.
  Residues:
  tcgaaagaac cattcgattt tcccatggca gctgagaata ttngtaggag acctgctaga 60
  gagatagtcg taacaatttc cgtaggtgaa cctgcggaag gatcattgtc gtttccttag 120
  aggacagacc cgcgaacatg ttatcattgc tttcttcaag ccgggcggtg catcgtgccc 180
  cacggggcag gttgatctgt tctgcttctt ggcatgtgcc cgtcttttgc catccccttt 240
  gtggtcgatt gcttgagcgg tgtactgcca aaacaacaaa aacggcgctt ttaagtgtca 300
  aggatcattt attgaatgaa agggggacat cccgccacaa aatgagtggg gggagaagtg 360
  cccctttgcc ttctaacaag aacgactctc ggcaacggat atcttggctc ctgtcacgat 420
  gaagaacgta gcgaaatgcg atagttggtg tgaattgcag aatcccgtga atcatcgagt 480
  ttttgaacgc aagttgcgcc cgaggccatt cggccaaggg cacgtctgcc tgggcgtcac 540
  gcttagcgtc gccctcccca ggtcctcgtg ttttcgaacc gaggccgagg gagagcggag 600
  gactggcctt cggtgtcgct ttcatcggcg tcgtcggctg aaactttcgg ctcacgatct 660
  gttgtgcagc acaacaagcg gtggatttcc agtgagttgt tgtgtttcac gtggtcaaag 720
  ggccatggga ctcgaggcaa ggttctcatt ttcttgcctt agctttgcga ccccaggtca 780
  ggcgagacta cccgctgagt ttaagcatat caataagcgg aggatcttgc tgaaaaactc 840
  gaaccnatcc sggagatccg gcggccgctc tccctatagg gtgagtcga 889
  Sequence Number (ID): 15
  Length: 1008
  Molecule Type: DNA
  Features Location/Qualifiers:
    - source, 1 .. 1008
      > mol_type, genomic DNA
      > note, matK8_Assembly
      > organism, Nymphaea sp.
  Residues:
  atttcccttt ttagaggaca aattatcaca tttatattat gtttcagata tactaatacc 60
  ctacccaatc catctggaaa tcttgcttca aactcttcgc actcggatac gagatgctcc 120
  ttctttgcat ttattgagat gttttctaca tgagcatcat aattggaata gccttattac 180
  ttctacttca aataaatcca tttccatttt ttcaaaggaa aatcaaagat tattcttgtt 240
  cttgtataat tctcatgtat atgaatgcga atccgtatta gttttccttc gtaaacaatc 300
  ctctcattta cggtcaatat cttctctagc ctttcttgag agaacacatt tttatggaaa 360
  aataaaacat cttgtagtga cgcctcgtaa tgattctcaa aggaccctgc ccctctggtt 420
  cttcaaagaa cctttgatgc attatgttag gtatcaagga aaatcaatta tggcttcaag 480
  gtgtactaat ttactgatga agaaatggaa atattacctt gtcaatttct ggcaatgtca 540
  ttttcactta tggtctcaac cgggtaggat ccatataaat gaattatcca atcattcttt 600
  ctattttctg ggctatcttt taggtgtacg actaacgcct tgggtgataa ggagtcaaat 660
  gctagagaat tcatttatga tcgatactgc tattaagaga ttcgatacaa tagtcccaat 720
  ttttcctctg attggatcgt tggttaaagc taaattctgt aacgtatcag ggtatcctat 780
  tagtaagtca gtctgggccg attcgtcgga ttctgatatt attgctcgat tcgggtggat 840
  atgcagaaat ctctctcatt atcacagcgg atcctcaaaa aaacacagtt tgtgtcgaat 900
  aaagtatata cttcgacttt cgtgtgctag aactctagct cgtaaacata aaagtacggt 960
  acgcgcaatc tgtaagagat taggttcaaa actattggaa gagttcct 1008
  Sequence Number (ID): 16
  Length: 977
  Molecule Type: DNA
  Features Location/Qualifiers:
    - source, 1 .. 977
      > mol_type, genomic DNA
      > note, matK11_Assembly
      > organism, Nymphaea sp.'
  Residues:
  atactaatac cctacccaat ccatctggaa atcttgcttc aaactcttcg cactcggata 60
  cgagatgctc cttctttgca tttattgaga tgttttctac atgagcatca taattggaat 120
  agccttatta cttctacttc aaataaatcc atttccattt tttcaaagga aaatcaaaga 180
  ttattcttgt tcttgtataa ttctcatgta tatgaatgcg aatccgtatt agttttcctt 240
  cgtaaacaat cctctcattt acggtcaata tcttctctag cctttcttga gagaacacat 300
  ttttatggaa aaataaaaca tcttgtagtg acgcctcgta atgattctca aaggaccctg 360
  cccctctggt tcttcaaaga acctttgatg cattatgtta ggtatcaagg aaaatcaatt 420
  atggcttcaa ggtgtactaa tttactgatg aagaaatgga aatattacct tgtcaatttc 480
  tggcaatgtc attttcactt atggtctcaa ccgggtagga tccatataaa tgaattatcc 540
  aatcattctt tctattttct gggctatctt ttaggtgtac gactaacgcc ttgggtgata 600
  aggagtcaaa tgctagagaa ttcatttatg atcgatactg ctattaagag attcgataca 660
  atagtcccaa tttttcctct gattggatcg ttggttaaag ctaaattctg taacgtatca 720
  gggtatccta ttagtaagtc agtctgggcc gattcgtcgg attctgatat tattgctcga 780
  ttcgggtgga tatgcagaaa tctctctcat tatcacagcg gatcctcaaa aaaacacagt 840
  ttgtgtcgaa taaagtatat acttcgactt tcgtgtgcta gaactctagc tcgtaaacat 900
  aaaagtacgg tacgcgcaat ctgtaagaga ttaggttcaa aactattgga agagttcctt 960
  acagaggaac aagaaat 977
END
```

1-10. (canceled)

11. An identification method of two parents of a Nymphaea hybrid based on sequences of internal transcribed spacer (ITS) and matK, comprising the following steps:
(i) construction of a database for each of nucleotide sequences ITS and matK of Nymphaea: downloading existing nucleotide sequences of ITS and matK of Nymphaea; conducting stringent filtration to retain one sequence for each “species unit”, namely species/subspecies/variety/form/hybrid of the sequences of ITS and matK; conducting alignment, adjusting sequences with inconsistent sequence directions, and then conducting re-alignment to obtain the database for each of nucleotide sequences ITS and matK of Nymphaea; and arranging a sequence name for each of finally obtained sequences in a format of “species name/ACCESSION of sequence”; and
(ii) construction of an ITS monoclonal sequence and an matK nucleotide sequence of a species for parents to be determined of the Nymphaea: extracting a genomic DNA of the Nymphaea of the parents to be determined; designing primers; conducting PCR sequence amplification; conducting Sanger sequencing; conducting sequence alignment and constructing a neighbor-joining tree based on a genetic distance, and then aligning sequences of ITS and matK obtained by monoclonal sequencing with the respective database; checking specific loci to obtain species information of male and female parents of the Nymphaea hybrid,
wherein the construction of the database for each of the nucleotide sequences ITS and matK of the Nymphaea specifically comprises the steps of:
(A) searching a nucleotide database of the National Center of Biotechnology Information (NCBI, www.ncbi.nlm.nih.gov/nucleotide) with syntaxes “((Nymphaea[Organism]) AND 5.8S [Title]) NOT PREDICTED[Title]” and “((Nymphaea[Organism]) AND matK[Title]) NOT PREDICTED[Title]”, to obtain all data of the sequences of ITS and matK of the Nymphaea; acquiring 59 published chloroplast genomes of the Nymphaea with a syntax “chloroplast, complete genome[Title] OR plastid, complete genome[Title]) AND Nymphaea[Organism]”, and extracting a matK sequence in the chloroplast genomes;
(B) conducting stringent filtration to retain one sequence for each “species unit”, namely species/subspecies/variety/form/hybrid of the sequences of ITS and matK; and
(C) conducting alignment, adjusting the sequences with inconsistent sequence directions, and then conducting re-alignment to obtain the database of the nucleotide sequences ITS and matK of the Nymphaea;
wherein the stringent filtration specifically comprises the steps of:
(a) removing a species whose species name comprises unverified, sp., and cf., retaining a subspecies, a variety, a form, and a clear hybrid that are included in the species, which are referred to as “species units”, and regarding each of an isolate, a voucher, a genotype, and a strain as a cultivar and classifying the cultivar into each “species unit”;
(b) if the “species unit” has only one sequence, omitting the filtration; after the filtration is conducted, retaining only one sequence for each “species unit”;
(c) if a sequence of the cultivar in step a is quite different from that of the “species unit”, removing the sequence of the cultivar preferentially;
(d) removing a sequence with a significantly high difference in a coding region 5.8S rRNA preferentially; removing a sequence having an unknown base and a degenerate base preferentially; and retaining a longer sequence preferentially;
(e) using the matK sequence extracted from the chloroplast genomes only as a supplement; when the matK sequence obtained by sequencing already has the “species unit”, discarding the matK sequence extracted from the chloroplast genomes; and
(f) retaining a sequence as a sequence that best represents the “species unit”, that is, a sequence being closest to a consensus sequence obtained from multiple sequences;
wherein the construction of an ITS monoclonal sequence and the matK nucleotide sequence of the species for parents to be determined of the Nymphaea in step (ii) specifically comprises the steps of:
(a′), designing an ITS primer suitable for the Nymphaea using conserved nucleotide sequences of 18S, 5.8S, and 25S rRNA of the Nymphaea and referring to a general plant barcode ITS primer; and designing a matK primer suitable for the Nymphaea using a conserved nucleotide sequence of matK of the Nymphaea and referring to a general plant barcode primer;
(b′) extracting the genomic DNA of the Nymphaea of the parents to be determined;
(c′) conducting PCR amplification using the genomic DNA of the Nymphaea of the parents to be determined obtained in step (b′) as a template and using the primers obtained in step a, to obtain amplification products of the sequences of ITS and matK, respectively;
(d′) ligating the amplification product of the ITS sequence obtained in step (c′) using a pLB zero-background rapid cloning kit, and transforming into DH5α competent cells; conducting PCR amplification on an obtained selected bacterial plaque through a carrier primer, to obtain a PCR amplification product of the ITS sequence by cloning amplification; and
(e′), subjecting the amplification product of the matK sequence obtained in step (c′) and the PCR amplification product of the ITS sequence by cloning amplification obtained in step (d′) to sequencing with a Sanger sequencer, and conducting sequence assembly on obtained sequencing results to obtain the matK sequence and the monoclonal ITS sequence for the parents to be determined of the Nymphaea; wherein
the primers comprise:
ITS-5, with a nucleotide sequence of AGTCGTAACAAGGTTTCCGT;
ITS-3, with a nucleotide sequence of TAGTAACGGCGAGCGAACC;
matK-5, with a nucleotide sequence of CGTACCGTACTTTTATGTTTACGAG; and
matK-3, with a nucleotide sequence of ACCCAATCCATCTGGAAATCTTGCTTC.
